(19) United States
(12) Patent Application Publication
(10) Pub. No.: US 2004/0044210 A1
(43) Pub. Date: Mar. 4, 2004

(54) Chiral nanotubes

(76) Inventor: Hicham Fenniri, West Lafayette, IN (US)

Correspondence Address: BARNES & THORNBURG, 11 SOUTH MERIDIAN, INDIANAPOLIS, IN, 46204

(21) Appl. No.: 10/630,574

(22) Filed: Jul. 30, 2003

Related U.S. Application Data
(60) Provisional application No. 60/399,951, filed on Jul. 30, 2002.

(51) Int. Cl. C07D 241/36; C07D 239/70

(52) U.S. Cl. 544/245; 544/338

(57) ABSTRACT

Nanotubes having synthetic receptors, and processes for their formation are described. In addition, nanotubes that form complexes with promoters, and processes for forming such complexes are described.

### CROSS-REFERENCE TO RELATED APPLICATIONS

The present application claims the benefit of provisional patent application Serial No. 60/399,951, filed on Jul. 30, 2002, the entire description of which is incorporated herein by reference. Cross reference is made to copending U.S. patent application Ser. No. 10/050292, entitled “Method and Associated Compounds for Forming Nanotubes,” filed on Jan. 16, 2002.

The invention described herein was made with government support under grant number AI 36624 and contract number NO1-CO-56000 awarded by the National Institutes of Health. The Government may have certain rights in the invention.

### FIELD OF THE INVENTION

The invention described herein pertains to nanotubes, and processes for preparing nanotubes. In particular, the invention pertains to nanotubes that are assembled from nanotube monomers that are not covalently bound to each other.

### BACKGROUND OF THE INVENTION

As the limits in miniaturization of various computer and biological technologies appear on the horizon, new generations of nano-sized devices are required. For example, the world of electronics and storage technology is constantly pushing into the nanoscale of components and architecture. Nanodevices include single electron transistors, molecular wire crossbar memories, nanoscale patterned magnetic arrays, and nanotubes. Nanotubes in particular are useful in information storage, in chemical storage, as channels for ion transport in batteries or living cells, as opacity varying electrodes in optical modulation devices or optical switches, in display technology, as molecular wires, and for the piezoelectric generation of electricity, among other applications.

New nanostructures in the form of nanotubes, where such nanotubes form spontaneously, or are induced to form under predetermined conditions provide current technologies with novel components.

### SUMMARY OF THE INVENTION

Nanotubes that may form spontaneously from nanotube monomers are described herein. The nanotubes include covalently linked synthetic receptors. The nanotubes can be formed by a network of hydrogen bonds between the nanotube monomers. In addition, the nanotubes are formed by the favorable stacking interactions of the ring motives.

In some embodiments the synthetic receptors are chiral; in other embodiments the synthetic receptors are achiral. The synthetic receptors present on the nanotubes are capable of associating, interacting, complexing, or binding with promoters. In some embodiments, the promoters are chiral; in other embodiments the promoters are achiral.

In some embodiments, the promoters described herein are capable of inducing the formation of nanotubes from nanotube monomers; in other embodiments the promoters are capable of stabilizing pre-existing nanotubes. In some aspects, the promoters described herein are also capable of inducing optical activity in a racemic or achiral mixture of pre-existing nanotubes. In other aspects, the promoters described herein are also capable of inducing the formation of optically active solutions of nanotubes from nanotube monomers.

Nanotube monomers are illustratively compounds having a heterobicyclic core that includes hydrogen bond donor and hydrogen bond acceptor groups, such as the compounds of formulae I and II:

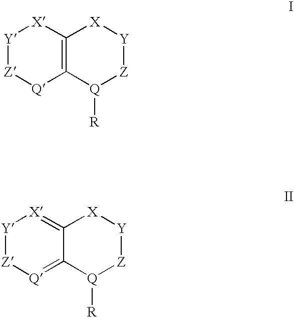

The groups X, X′, Y, Y′, Z and Z′ are each independently selected from hydrogen bond donors and hydrogen bond acceptors. The groups Z and Z′ may also independently represent a single or a double bond connecting Y and Q, and Y′ and Q′, respectively. The groups Q and Q′ are each independently selected from carbon and nitrogen; and R is a synthetic receptor, or a derivative thereof Illustratively, Q and Q′ are each independently selected from —N—, —NH—, ═N—, —CH—, —CH₂—, and ═CH—. The groups X, X′, Y, Y′, Z and Z′ are selected such that adjacent monomers in the nanotube architecture may for hydrogen bonds.

Exemplary hydrogen bond donors include divalent radicals such as the following formulae:

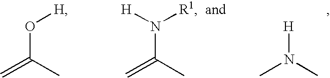

where R¹ is hydrogen or alkyl. Exemplary hydrogen bond acceptors include divalent radicals such as the following formulae:

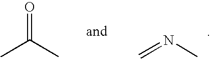

It is appreciated that depending upon the hydrogen bond donor or hydrogen bond acceptor that is selected for the various groups X, X′, Y, Y′, Z and Z′, when either of Z or Z′ does not represent a single or a double bond, the connectivity between adjacent groups, such as X and Y, Y and Z, Z and Q, X′ and Y′, Y′ and Z′, or Z′ and Q′, is a single or a double bond. It is therefore understood that the bonding between those groups is illustrated schematically by formulae I and II. For example, when X is the group —C(O)—, and Y is the group ═N—, the grouping X-Y corresponds to —C(O)—N═. In another illustrative example, when Z′ is the group ═C(OH)— and Q′ is the group ═C(NHR¹)—, the grouping Z′-Q′, is —C(OH)═C(NHR¹)—.

Synthetic receptors include the formula —(CH₂)ₙ—R′, where n is an integer selected from 2, 3, 4, and 5, and R′ is selected from the group of crown ethers, cryptands, cyclodextrins, amino acids, peptides, diamines, triamines, and derivatives thereof. In some aspects, the crown ether is aminobenzo-18-crown-6, the amino acid is lysine, and the poly amine is 1,5-diaminopentane

Promoters include amines and amino acids, including amino acids having a primary amine functionality. The promoters may be achiral or chiral, and include alpha amino acids, where the amino acid is substituted with alkyl, optionally-substituted aryl, optionally-substituted arylalkyl, thioalkyl, alkylthioalkyl, hydroxyalkyl, alkoxyalkyl, aminoalkyl, alkylaminoalkyl, dialkylaminoalkyl, or a group —(CH₂)ₘ—R″, where m is an integer selected from the group consisting of 1, 2, 3, 4, and 5; R″ is —CO₂R², —CONR³R⁴, or —NR⁵C(NR⁶)NR³R⁴, and R², R³, R⁴, R⁵, and R⁶ are each independently selected from the group consisting of hydrogen, alkyl, and optionally-substituted arylalkyl.

Illustratively, the promoter is alanine, leucine, 2-butyl-2-aminoethanoic acid, phenylalanine, 2-(naphth-2-ylmethyl)-2-aminoethanoic acid, methionine, serine, glutamic acid, and glutamine.

Processes for forming nanotubes are described herein. These processes include processes for forming achiral and chiral nanotubes, and optically active solutions of nanotubes. In some embodiments, the nanotubes form spontaneously from nanotube monomers; in other embodiments, the nanotube formation is facilitated by the addition of a promoter. Processes for forming optically active solutions of nanotubes include the introduction of optically active solutions of promoters or homochiral promoters.

Processes are also described for forming dilute solutions of achiral and chiral nanotubes, and dilute optically active solutions of nanotubes. Processes for stabilizing solutions of achiral or chiral nanotubes to dilution are also described.

### BRIEF DESCRIPTION OF THE DRAWINGS

FIG. 1 shows a ring motif formed by compound 1 (n=2).

FIGS. 2A and 2B show two perspectives of a stacking motif formed by compound 1 (n=2).

FIG. 3 shows the CD spectra of nanotubes formed from compound 1, n=2 [0.04 mM] and either L-Ala [0.4 mM] (◯) or D-Ala (●) [0.4 mM], recorded continuously until the induced circular dichroism (ICD) stabilized, generally within 24 h after mixing.

FIG. 4 shows a schematic illustration of the helical arrangement of synthetic receptors on a nanotube.

### DETAILED DESCRIPTION OF THE INVENTION

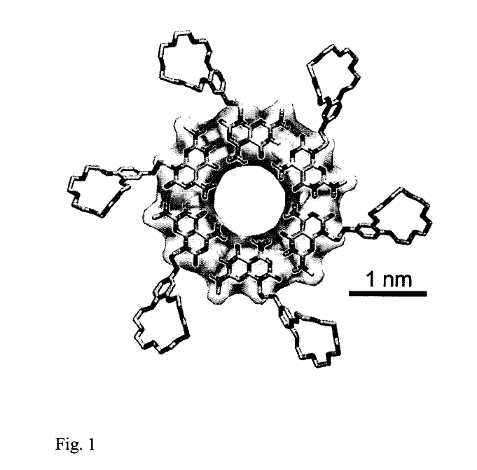

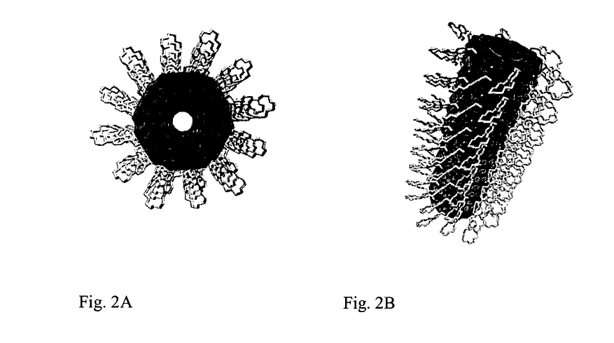

Nanotubes are formed from two structural components. The monomers are arranged in rings or donut shapes, as illustrated by the embodiment depicted in FIG. 1. Further, the rings or donut shapes are arranged in stacks, as illustrated by the two perspective views of the embodiment in FIGS. 2A and 2B. The views illustrated in FIG. 1 and in FIGS. 2A and 2B were generated by Macromodel 7.2 and VMD. In some embodiments the ring motif forms first; in other embodiments, the stacked motif forms first; and in yet other embodiments the ring and stack motives form contemporaneously during the assembly of the nanotubes described herein. Stacking of the ring motives produces thereby a tubular architecture that possesses a central unoccluded pore running the length of the stack. It is appreciated that the size of the central pore may be advantageously pre-determined by the appropriate selection of nanotube monomers. It is further appreciated that the outer diameter of the nanotube is also pre-determined by the appropriate selection of nanotube monomers.

In some embodiments, the central pore is more hydrophilic in character than the outer surface. In other embodiments, the central pore is more hydrophobic in character than the outer surface. It is appreciated that the relative hydrophobicity or hydrophilicity of the nanotube pore and the nanotube exterior are advantageously predetermined by the appropriate selection of nanotube monomers. It is further appreciated that in variations of the nanotubes described herein, the nanotube monomer may be modified such that following assembly the resulting exterior surface of the nanotube or the central pore may be made more or less hydrophilic by the appropriate selection of nanotube monomers. It is further appreciated that the relative hydrophobicity or hydrophilicity of the exterior surface of the nanotubes described herein are optionally modified following the interaction of various promoters with the synthetic receptors.

The ring motives are formed from monomers that are associated with each other through a network of hydrogen bonds. As illustrated by the exemplary embodiment in FIG. 1, each monomer includes a number of hydrogen bond donor and hydrogen bond acceptor groups. The monomers are arranged in the ring motif in a configuration that allows for the formation of hydrogen bonds between these various donor and acceptor groups. In addition, these ring motives stack to form columnar arrangements. Without being bound by theory, it is believed that the stacked arrangement of the ring motives is stabilized by a series of hydrophobic and/or π-stacking interactions.

At higher concentrations, the nanotubes described herein form spontaneously from nanotube monomers in solution. At lower concentrations, the nanotubes described herein form following the introduction of a suitably selected promoter. Promoters that are capable of associating, interacting, complexing, or binding with the synthetic receptor present on existing nanotubes can stabilize the nanotube architecture to changes in conditions, such as increases in temperature, decreases in concentration, and changes in solvent composition, such as increases or decreases in solvent polarity.

In addition, promoters that are capable of associating, interacting, complexing, or binding with the synthetic receptor present on nanotube monomers can induce the formation of nanotubes. In the case where the promoter is introduced to induce the self-assembly of the nanotube, it is understood that the promoter facilitates assembly of the nanotube under conditions where nanotube formation will likely not occur in the absence of the promoter due to inappropriate conditions for nanotube formation such as temperature, concentration, solvent composition, and the like.

The presence of nanotubes forming in solutions can be measured and monitored by any of a variety of techniques including, but not limited to, transmission electron microscopy (TEM), dynamic light scattering (DLS), small angle X-ray scattering (SAXS), and circular dichroism (CD) studies.

As indicated herein, nanotubes described herein can include synthetic receptors. Promoters added to solutions of nanotube monomers or to solutions of already existing nanotubes interact with such synthetic receptors. This interaction provides the mechanism for nanotube formation in the case of dilute solutions where nanotubes do not form spontaneously, and for nanotube stabilization in cases where the nanotubes form spontaneously, as described herein.

In one embodiment, the nanotubes described herein include a plurality of synthetic receptors. The synthetic receptors are covalently bound to certain nanotube monomers. In one aspect, the nanotubes are assembled from a plurality of monomeric units having the same chemical structure. In this configuration, each monomer is covalently linked to a synthetic receptor. In another aspect, the nanotubes are assembled from monomers having two or more chemical structures. In this configuration, some or all of the monomers have a covalently linked synthetic receptor. It is appreciated that such synthetic receptors may be the same or different depending upon the chemical structure of each monomer used in nanotube formation.

Depending upon the nature of the nanotube monomers and the covalently attached synthetic receptors, the nanotubes described herein can exhibit or display a helical or helicoidal secondary structure. Without being bound by theory, such helical patterns may be due to steric or electronic effects exerted by the synthetic receptors or the synthetic receptors once complexed or bound with promoters, arising from the proximity and location of the synthetic receptors on the outer surface of the nanotubes described herein.

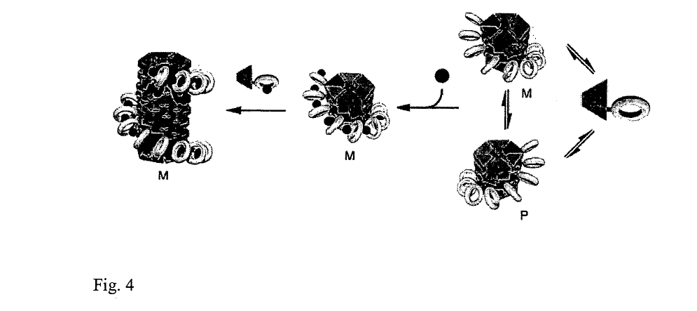

In one embodiment, the synthetic receptors are arranged in a helical or helicoidal pattern on the outer surface of the nanotube. This helical or helicoidal pattern or arrangement of the synthetic receptors may is illustrated in the schematic nanotube shown in FIG. 4. For clarity, only a single synthetic receptor per ring motif is shown in FIG. 4. Thus, depending upon the multiplicity of monomers having synthetic receptors that form the ring motif, a different number of helical bands may encircle the nanotubes described herein. For example, in the nanotubes of compound 1, n=2 depicted in FIG. 2A and 2B, the ring motif is formed from six monomer units. Hence, six helices spiral about or encircle the longitudinal axis of the nanotubes. In addition, the helical or helicoidal patterns arranged on the exterior if the nanotubes described herein can be chiral. Nanotubes that form right handed helicies are denoted as P nanotubes. Nanotubes that form left handed helicies are denoted as M nanotubes. The handedness of the helices formed on the nanotubes described herein is determined by viewing the nanotube from the end, transverse to the nanotube central axis.

The helical pattern imparts to the nanotubes a chirality or handedness. Such chirality may be formed even in the case of nanotubes that include achiral synthetic receptors such as compound 1, n=2 shown in FIGS. 2A and 2B, where the synthetic receptor is an achiral crown ether derivative. However, solutions of such nanotubes are typically racemic because there are an equal number of M and P nanotube configurations formed as a statistical mixture in the solution. In another embodiment, the synthetic receptors are achiral, and are not arranged in any discernable pattern. Such nanotubes are achiral.

In another embodiment, the synthetic receptors are chiral. In one aspect, the nanotubes are formed from a homochiral plurality of monomers that include such chiral receptors, and are also chiral. Solutions of such nanotubes are optically active when the relative population of monomers having one chirality exceeds the relative population of monomers having the other chirality. It is therefore appreciated that nanotubes formed from a mixture of monomers having different chiralities may be or may not be chiral depending on the nature and relative populations of such chiral monomers.

In addition, to the stabilization and induction of formation capabilities of promoters in the present invention described herein, in some embodiments chiral promoters also stabilize, induce, or alter the chirality of the nanotubes described herein.

In some embodiments, chiral promoters are capable of transferring their chirality to the nanotubes described herein, both those that are pre-existing and those whose formation is induced by the introduction of the promoter. A transfer of chirality from the promoter to the nanotube occurs in such a way that like-handed promoters tend to induce or stabilize the same handedness in the resulting chiral nanotubes.

In some embodiments where a chiral promoter is absent, and where the synthetic receptor is achiral, the nanotubes are achiral. In other embodiments where a chiral promoter is absent, and where the synthetic receptor is achiral, the nanotubes are chiral, but exist as a racemic mixture in solution. It is appreciated that the nature and structure of the monomer forming the nanotube, the solvent, and the temperature alone or in combination determine whether the nanotube is achiral or chiral yet present in solution as a racemic mixture.

In one embodiment, the nanotube is present in solution as a racemic mixture of M and P helicities, as illustrated in FIG. 4. Illustratively, the nanotubes are in equilibrium. Introduction of a chiral promoter can stabilize the architecture of the nanotubes having the complementary chirality or helicity. In addition, introduction of a chiral promoter can convert nanotubes having the opposite chirality or helicity into nanotubes having the complementary chirality or helicity. FIG. 4 shows the equilibrium between the M and P helicities, and further that the population of, for example, M helicities can be increased contemporaneously with a decrease in the population of P helicities upon binding of a suitably selected promoter (depicted by ● in FIG. 4). In one aspect, the M and P nanotubes can interconvert in this equilibrium. In another aspect, the equilibrium is shifted by disassembling one helical form, for example the P helicity illustrated in FIG. 4, into monomers that subsequently reassemble into the other helical for, illustratively the M helicity.

In one aspect, the promoter is introduced as a solution having optical activity, such that there is present in the promoter solution a preponderance of one chirality over the other. Such solutions can effect optical activity in the racemic solutions of existing nanotubes via chirality stabilization, chirality conversion, or symmetry-breaking processes, as described herein.

In some embodiments, the concentration of the monomer is too low for assembly of the nanotube. It is appreciated that the nature and structure of the monomer forming the nanotube determines the threshold concentration at which nanotubes may form from such monomers. Introduction of a suitably selected promoter induces the formation of nanotubes from these nanotube monomers under conditions that they will not spontaneously form.

In one aspect, the promoter is introduced as a solution having optical activity, such that there is present in the promoter solution a preponderance of one chirality over the other. Such solutions can effect the formation of nanotubes from the nanotube monomers as well as effect optical activity in the nanotube solutions. It is appreciated that the mechanism for induction of chirality may depend upon the nature of the monomer, the nature of the promoter, the temperature, and the solvent composition. Chirality induction may occur contemporaneously as the nanotubes assemble under the influence of the promoter. Alternatively, the promoter may first induce the formation of the nanotubes without the induction of a complementary chirality, then the existing nanotube-promoter complexes are converted into a preponderance of one chirality that corresponds to the preponderance of chirality exhibited by the optically active promoter via the chirality stabilization, chirality conversion, or symmetry breaking processes described herein.

In some embodiments, the presence of a promoter in the solution of nanotubes, whether pre-existing prior to introduction of the promoter or nanotubes whose formation was induced, stabilizes the nanotube architecture to increases in temperature, decreases in concentration, or changes in solvent composition, such as increases or decreases in solvent polarity.

It is appreciated that such induction, stabilization, and conversion properties of the promoters described herein used in conjunction with the nanotubes described herein are useful in various technological applications. It is further appreciated that depending on the nature of the promoter, the physical and chemical properties of the resulting nanotube-promoter complex may be designed or tailored by selecting appropriate promoters to impact desired properties such as charge, hydrophilicity, hydrophobicity, dipole, fluorescence, and the like. Chiral nanotubes described herein possess optical properties that are tunable or adjustable.

In one embodiment, the tunable nature of the nanotubes described herein can be adjusted in both magnitude and in direction. The tunable nature of the nanotubes may arise from modifications of properties, such as the chirality of the nanotubes, or the optical properties of the nanotubes. Introduction of a suitably selected promoter, or changing the relative concentrations of two or more promoters can form the basis for tuning the chiral or the optical properties of nanotubes described herein. In addition, changing the environment in which the nanotubes are formed, such as changing the concentration, temperature, or solvent composition, solvent polarity, or solvent ionic strength, can form the basis for tuning the chiral or the optical properties of nanotubes described herein.

It is understood that the processes for forming the nanotubes, as well as the tunable or adjustable optical properties of the nanotubes, described herein can be used advantageously in applications including chirotechnology, as described by Canary & Zahn, TRENDS Biotech., 19, 251-55 (2001); for the design of sensors, as described by Rivera et al., Angew. Chem. Intl. Ed. Engl., 39, 2130-32 (2000); chiral cholesteric phases, as described by Tanatani et al., J. Am. Chem. Soc., 123, 1792-93 (2001); catalysts, as described by Lorenzo et al., Nature, 404, 376-79 (2000); asymmetric synthesis of materials for electromagnetic and optoelectronic applications, as described by Akagi et al., Science, 282, 1683-86 (1998); information storage, as described by Iftime et al., J. Am. Chem. Soc., 122, 12646-50 (2000); display systems, as described by Feringa et al., Adv. Mat., 8, 681-84 (1996); photochromic materials, as described by Ichimura, Chem. Rev., 100, 1847-73 (2000); materials with unique chiral light-emitting and non-linear optical properties, as described by Verbiest et al., Science, 282, 913-15 (1998); and the like. The disclosures of the foregoing are incorporated herein by reference.

In some embodiments, the synthetic receptor is chiral. Under conditions, where nanotubes form spontaneously, the resulting nanotubes are chiral. Further, in cases where the synthetic receptor is homochiral, a solution of the resulting nanotubes is optically active and possesses CD activity. In cases where nanotube assembly is induced by a promoter, the introduction of either an achiral or chiral promoter may induce optical activity in the solution of resulting nanotubes.

In another embodiment, nanotubes are formed from chiral promoters, where the chiral promoters are present with a preponderance of one chirality thereby forming an optically active solution of nanotubes. The nature, handedness, or sign of this optical activity can be reversed upon the addition to the solution of nanotubes of a second chiral promoter, where the second chiral promoter is optically active. In one aspect, the second optically active chiral promoter is chemically identical to the first optically active chiral promoter. In another aspect, the second optically active chiral promoter is chemically different from the first optically active chiral promoter. It is appreciated that the optical activity can be reversed upon addition of a relative excess of the second promoter. The magnitude of the excess required is dependent upon the nature of the first and second promoters, and of the nanotube monomers and associated synthetic receptors.

In both processes, the induction of optically activity or a preponderance of one helicity over another into a solution of racemic nanotubes, or the formation of dilute solutions of optically active or racemic nanotubes, it is appreciated that depending upon the nature of the promoter and the nature of the nanotube monomer differential levels of synthetic receptor occupancy are required. In one embodiment, the majority of the synthetic receptors are associated with or complexed to a promoter.

Nanotube monomers that may be used in the present invention to form the nanotubes described herein include molecules that possess an array of hydrogen bond donor and hydrogen bond acceptor functional groups. These hydrogen bond forming functional groups are arranged such that two adjacent monomers in the ring motif portion of the nanotubes described herein display hydrogen bond donor and hydrogen bond acceptor functional groups in a manner that allows for the formation of hydrogen bonds between them.

An hydrogen bond donor is generally a functional group possessing an hydrogen that is capable of participating in an hydrogen bond with an hydrogen bond acceptor. Lewis acids and Bronsted-Lowry acids are also considered to be hydrogen bond donors within the meaning of the term as used herein.

An hydrogen bond acceptor is generally a functional group possessing an electron pair that is capable of participating in an hydrogen bond with an hydrogen bond donor. Lewis bases and Bronsted-Lowry bases are also considered to be hydrogen bond donors within the meaning of the term as used herein.

Some functional groups present on nanotube monomers described herein can be both hydrogen bond donors and hydrogen bond acceptors. For example, a carboxamide functional group accepts a hydrogen bond on the carboxyl oxygen atom and donates a hydrogen bond from the amine group hydrogen. Hydrogen bond donors and hydrogen bond acceptors may be present on a variety of functional groups, including but not limited to amine groups, hydroxy groups, imine groups, carbonyl groups, carboxyl groups, such as acids, esters, amides, and guanides, sulfhydryl, groups, sulfinyl groups, sulfonyl groups, phosphinyl groups, phosphonyl groups, phosphoryl groups, and the like. As described herein, the ring motif is formed by the interaction of the hydrogen bond donor groups with the hydrogen bond acceptor groups from adjacent nanotube monomers, such as a nanotube monomer having an hydrogen on an amide group nitrogen forming an hydrogen bond to the oxygen of carbonyl group or a carboxyl group present on the adjacent nanotube monomer.

In one embodiment, the nanotube monomer includes compounds that have a heterobicyclic core having the formula I:

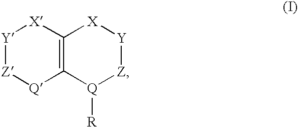

where X, X′, Y, Y′, Z, and Z′ are hydrogen bond donors or hydrogen bond acceptors, Q and Q′ are carbon and nitrogen atoms, that may be optionally substituted, and the group R is a synthetic receptor, or a derivative thereof.

In other embodiments, monomers include compounds that have a heterobicyclic core having the formula II:

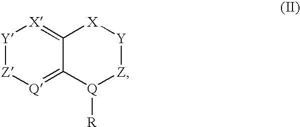

where X, X′, Y, Y′, Z, and Z′ are hydrogen bond donors or hydrogen bond acceptors, Q and Q′ are carbon and nitrogen atoms, that may be optionally substituted, and the group R is a synthetic receptor, or a derivative thereof.

In still other embodiments, monomers include compounds of the formulae I and II where Z is an hydrogen bond donor, an hydrogen bond acceptor, or Z represents a single or a double bond connecting Y and Q; and Z′ is an hydrogen bond donor, an hydrogen bond acceptor, or Z′ represents a single or a double bond connecting Y′ and Q′.

In some aspects of formulae I and II, where the nanotube is assembled from a single monomer selected from formulae I or II, when X is an hydrogen bond donor, X′ is an hydrogen bond acceptor; when X is an hydrogen bond acceptor, X′ is an hydrogen bond donor; when Y is an hydrogen bond donor, Y′ is an hydrogen bond acceptor; when Y is an hydrogen bond acceptor, Y′ is an hydrogen bond donor; when Z is an hydrogen bond donor, Z′ is an hydrogen bond acceptor; and when Z is an hydrogen bond acceptor, Z′ is an hydrogen bond donor. However, it is appreciated that mixtures of monomers selected from the compounds of formulae I and II may be used to assemble the nanotubes described herein.

In one embodiment, the nanotube monomer is a compound having the formula III.

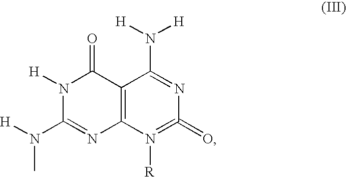

where the group R is a synthetic receptor, or a derivative thereof. In this embodiment, nanotubes are formed by the monomer arranging itself into hexameric rings, each constructed by a series of 18 hydrogen bonds, and the arrangement of these hexameric rings into columnar stacks.

In one aspect, the group R is a crown ether, a cryptand, a cyclodextrin, an amino acid, a polyamine, and the like, or a derivative thereof. Crown ether derivatives and cryptand derivatives that can serve as synthetic receptors include, but are not limited to, 18-crown-6-ethers and derivatives thereof, and the like. Amino acids that can serve as synthetic receptors include but are not limited to lysine, aspartic acid, glutamic acid, glutamine, arginine, and the like. Polyamine derivatives that can serve as synthetic receptors include but are not limited to ethylene diamine, diethylene triamine, triethylenetetraamine, and the like, and various homologs thereof, such as propylene diamine, butylene diamine, pentylene diamine, and the like.

Examples of such synthetic receptors include the following derivatives of formula III:

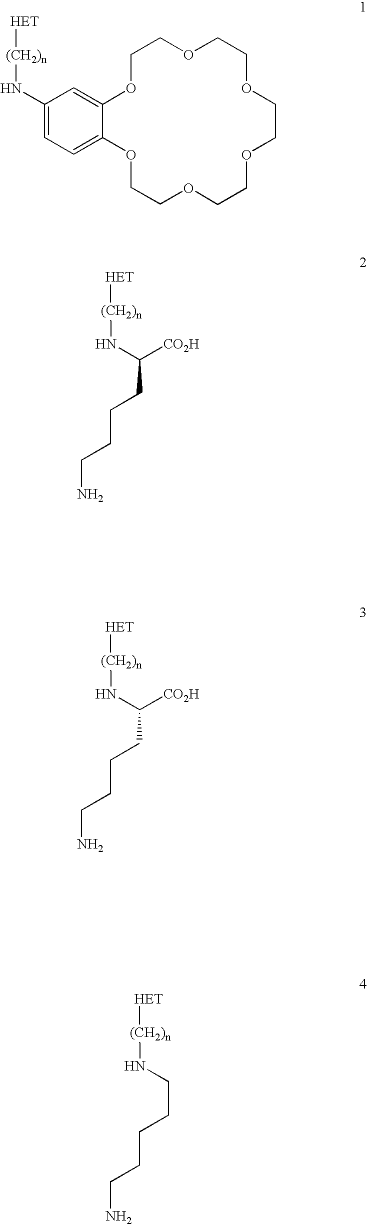

where HET is the heterobicyclic core of formula III, and n is an integer selected from 2, 3, 4, and 5. It is appreciated that the protonation state of the various nitrogen atoms, and the deprotonation state of the various carboxyl groups present in the nanotube monomer depends upon the pH under which the nanotubes described herein are assembled. Compounds 1, 2, 3, and 4 are prepared from standard procedures described in Fenniri et al., J. Am. Chem. Soc. 2000, 123, 3854-55 and in Fenniri et al., Proc. Natl. Acad. Sci. USA 2002, 99, 6487-92, the disclosure of which is incorporated herein y reference.

In one aspect, the synthetic receptor R is an 18-crown-6-ether derivative, such as 4-aminobenzo-18-crown-6-ether (18C6), the nanotubes formed therefrom have a concentration-dependent hydrodynamic radius in the range of about 10 to about 100 nm, and illustratively an average hydrodynamic radius of about 33 nm for compound 1, n=2, as determined by DLS. In addition, the central pore that is formed as a consequence of the arrangement of monomers 1 has a diameter of about 4 nm. In this illustrative embodiment, the crown ether synthetic receptor provides about 328 A³ of space that may be occupied by a promoter.

At higher concentrations, such as concentrations that are about 1 mM or greater, in solvents such as methanol, water, and the like, nanotubes formed from nanotube monomers such as compound 1 self assemble. Nanotubes can also self assemble at other concentrations, such as concentrations of about 0.04 mM or higher. At lower concentrations, such as concentrations of less than about 0.04 mM, nanotube monomers such as compound 1 assemble into nanotubes following the addition of a suitable promoter. Promoters that are capable of associating, interacting, complexing, or binding with the synthetic receptors present on the nanotube described herein facilitate the formation of nanotubes at lower concentrations. The absence of nanotubes, and therefore the presence of unassembled nanotube monomers, such as compound 1 is shown by TEM and DLS. Following the introduction of a promoter capable of facilitating the assembly of the nanotubes such as compound 1, these same techniques are used to indicate the presence of nanotubes.

In one embodiment, the promoter is an amino acid. In one aspect, the promoter is an amino acid including the naturally-occurring amino acids, such as glycine, alanine, phenylalanine, serine, cysteine, lysine, aspartic acid, tryptophan, histidine, and the like. In another aspect, the amino acid promoters include any homologous amino acid, such as alpha, beta, or gamma amino acids, including aminoethanoic acid, aminopropanoic acid, and the like. Such amino acid homologs may be optionally substituted, such as 2-substituted aminoethanoic acids, 2-substituted, 3-substituted, and 2,3-disubstituted aminopropanoic acids, and other homologs, such as optionally substituted aminobutanoic acids, optionally substituted aminopentanoic acids, and the like.

In another embodiment, the promoter is an amino acid having a chiral center. Such chiral promoters may be used to promote nanotube assembly using a single enantiomer, a homochiral promoter, or an optically active solution of the promoter, where one enantiomer is present in higher amounts than the other enantiomer. It is appreciated that depending upon the nature of the nanotube monomer and the covalently attached synthetic receptor, higher or lower enantiomeric excesses in the promoter used to induce chirality on the nanotube may be required. The nature of the substitution in forming the chiral center on the amino acid may include groups such as halogen, hydroxy, alkoxy, haloalkoxy, amino, alkylamino, dialkylamino, thio, alkylthio, optionally-substituted aryl, alkyl, haloalkyl, haloalkoxyalkyl, optionally-substituted arylalkyl, hydroxyalkyl, alkoxyalkyl, thioalkyl, alkylthioalkyl, and a group —(CH₂)ₘ—Z, where Z is —CO₂R¹, —CONR²R³, and the like, and m is an integer, illustratively selected from 1 to about 5.

In one aspect, the amino acid promoter is illustratively selected from alanine, methionine, leucine, phenylalanine, naphth-2-ylmethylglycine, n-leucine, serine, glutamic acid, glutamine, and the like. In another aspect, the promoter is an achiral amino acid, such as glycine, and the like.

In another aspect, the chiral promoter is an amino acid, amino alcohol, or an amine selected from dimethylalanine, homoserine, threonine, aspartic acid, asparagine, valine, 2-aminobutane, 2-aminopropanol, 3-aminopropanol, 3-aminobutanol, and the like.

In another embodiment, where the nanotube monomer is compound 1, the promoter is illustratively a primary alpha amino acid having a small, hydrophobic, and aromatic or aliphatic side chain, such as alanine, methionine, leucine, phenylalanine, and naphth-2-ylmethylglycine. In another embodiment, the promoter is illustratively a primary alpha amino acid selected from n-leucine, serine, glutamine, and glutamic acid. In another embodiment, the promoter is an achiral amino acid such as glycine.

In another embodiment, an enantiomeric excess of one helical form of the nanotubes described herein over the other is generated. The chiral promoter is illustratively added to a solution containing the chiral nanotubes at a concentration of about 5 to about 50-fold higher than the theoretical concentration of nanotube monomers in the solution. For example, a 0.046 mM solution of compound 1, n=2 in methanol forms an enantiomeric excess upon addition of a 2.8 mM solution of L-Ala in methanol. The transition from a racemic to an optically active solution of nanotubes can be monitored by CD. In some aspects, the transition from racemic to optically active nanotubes is rapid. It is appreciated that with certain nanotube monomers and certain homochiral promoters, a substantial number of the synthetic receptors, such as the crown ether receptors of compound 1, located on the nanotube monomers are bound to a promoter prior to the induction of homochirality or optical activity in the nanotube solution. In other embodiments, a lower population of synthetic receptors having a promoter bound or complexed thereto is necessary to induce homochirality or to generate an optically active solution of nanotubes.

In one embodiment, the nanotube formation or the optical activity exhibited by the nanotubes is reversible. The nanotube formation or the optical induction is disrupted upon the application of sufficient heat to break up the complex formed between the promoter and the nanotube. Further, the association of the monomers forming the nanotube may be disrupted upon the application of sufficient heat to disrupt the hydrogen bonding network and/or the favorable hydrophobic or π-stacking interactions that may stabilize the stack of ring motifs. Upon returning to the original temperature state, the nanotubes can reform. In addition, the complex between the nanotubes and the promoter may reform to again induce optical activity in the nanotube solution.

The nanotube formation or optical induction is also reversible upon the addition of a suitable complexing agent. The complexing agents that can bind the promoter with similar affinity to that exhibited by the nanotubes can disrupt the chiral induction of the nanotube caused by the promoter. For example, addition of a crown ether derivative to a solution of nanotubes formed from compound 1 and L-Ala causes the loss of the ICD. Other complexing agents that bind L-Ala or other promoters are contemplated herein.

It is similarly appreciated that changes in concentration may also disrupt either the complex between the nanotube and the promoter, or the association of monomers forming the nanotubes. As the concentration of the components is decreased, these interactions become less stable and may eventually cease. It is however appreciated that the presence of a promoter stabilizes the nanotube architecture in many cases. In another example of the reversible nature of the nanotube formation processes described herein, upon returning to the original concentration, the nanotube-promoter complex or the nanotubes themselves may reform.

It is similarly appreciated that changes in solvent composition, such as changes in the polarity of the solvent may also disrupt either the complex between the nanotube and the promoter, or the association of monomers forming the nanotubes. In another example of the reversible nature of the nanotube formation processes described herein, upon returning to the original solvent composition, the nanotube-promoter complex or the nanotubes themselves may reform.

The following examples further illustrate exemplified embodiments and aspects the invention. The examples illustrated herein are intended only to further describe the invention and should not be interpreted as limiting the invention.

### EXAMPLES

### Example 1

A 2.0 mM stock solution of compound 1 in MeOH and a 4.0 mM stock solution of the promoter in MeOH were premixed, placed in a quartz cuvette, and diluted with MeOH to a final concentration 0.04 mM 1 and 0.4 mM promoter. The CD spectra were recorded immediately at ambient temperature and periodically thereafter until the CD signal stabilized at the indicated wavelengths. CD measurements were recorded on a Jasco J810 CD spectropolarimeter.

The following table describes a series of promoters that when mixed with solutions of compound 1, n=2 induced chirality in the racemic mixture of helical forms.

| | Induced ellipticity (mDeg)^{(a)} | | |
|---|---|---|---|
| Promoter^{(b)} | 237 nm | 279 nm | 291 nm |
| 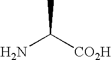 | −40 | −10 | +10 |
| 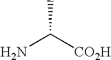 | +40 | +10 | −10 |
| 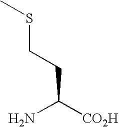 | −65 | −21 | +11 |
| 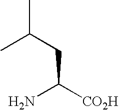 | −40 | −12 | +13 |
| 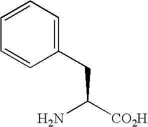 | −56 | −17 | +16 |
| 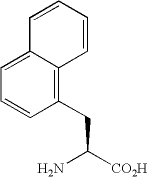 | −50 | −10 | +20 |
| 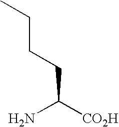 | −23 | −7 | +7 |
| 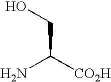 | −20 | −6 | +6 |
| 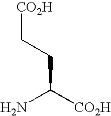 | −19 | −5 | +5 |
| 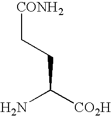 | −19 | −5 | +5 |

^{(a)}Maximum induced ellipticity (mDeg); the CD spectra were recorded continuously until stabilization of the induced circular dichroism (ICD), generally not more than 24 h after mixing.
^{(b)}[1, n = 2] = 0.04 mM, [promoter] = 0.4 mM.

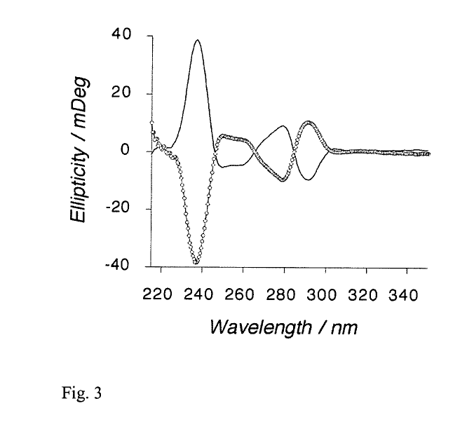

Promoters having the same absolute stereo configuration induced the same helicity in the solution of nanotubes formed from compound 1, n=2. The magnitude of the chiral induction was dependent upon the nature of the chiral promoter and its ability to complex with the nanotube bearing a crown ether synthetic receptor. A complete CD spectrum was obtained for the complex formed between compound 1 and L-Ala and compound 1 and D-Ala. The spectra are shown in FIG. 3. FIG. 3 illustrates that the two enantiomers induce opposite chiralily or helicity in the solution of pre-existing nanotubes formed from compound 1 (n=2). The induced CD spectra demonstrate that following the introduction of a homochiral promoter to the racemic mixture of nanotubes, a preponderance of one helicity is observed. The majority helix is the opposite when an homochiral promoter of the opposite enantiomer is introduced to a solution of nanotubes of compound 1.

### Example 2

A mixture of compound 1, n=2 [0.04 mM] and L-Ala [0.4 mM] in MeOH exhibited the induced CD spectral behavior as described in Example 1. The solution was heated to about 40° C. No change in the CD spectrum was observed. The sample was heated to 60° C. and the ICD decreased. Upon cooling the solution to 25° C, the ICD was 70% restored within a few minutes and completely restored within 24 h.

### Example 3

The conditions of Example 2 were repeated except with L-Ala [2.0 mM]. Upon heating to 60° C. and cooling to 25° C., the ICD was completely restored within a few minutes.

### Example 4

A mixture of compound 1, n=2 [0.04 mM] and L-Ala [0.4 mM] in MeOH was treated with 10 equivalents of D-Ala [0.4 mM]. The CD profile was inverted to match that obtained from a mixture of compound 1 [0.04 mM] and D-Ala [0.4 mM] in MeOH. In addition, the magnitude of the CD spectra were identical, indicating that the presence of L-Ala in the first solution did not influence the ICD following addition of the excess of D-Ala.

### Example 5

A solution of 1, n=2 [2.0 mM] in MeOH was treated with L-Ala (50 equivalents). The final concentration of 1 was 0.046 mM, and of L-Ala was 2.8 mM. CD indicated instantaneous formation of chiral nanotubes, showing a rate constant of k₀=0.48 s⁻¹ from racemic to chiral rosette nanotubes. The solution was diluted 50-fold. Both DLS and TEM indicated the persistence of the nanotubes.

A solution of 1, n=2 [0.04 mM] in MeOH was added to solution of L-Ala (50 equivalents). CD indicated instantaneous formation of chiral nanotubes despite the dilution.

A solution of 1, n=2 [0.04 mM] in MeOH was diluted with MeOH. CD activity was not observed, and the presence of nanotubes was not observed by DLS or TEM. Addition of L-Ala (10 equivalents) led to rapid host-guest complex formation. A typical ICD profile was observed after several hours, showing a rate constant of k₀=0.07 s⁻¹ from monomer to chiral rosette nanotubes. The final concentration of 1 was 0.04 mM, and of L-Ala was 2.4 mM.

### Example 6

Solutions of 1, n=2; 18C6; L-Ala; D-Ala; (L-Ala+18C6); (D-Ala+18C6); or (1, n=2+DL-Ala) did not exhibit any CD activity in the wavelength range of 200-350 nm.

### Example 7

Compounds 2 and 3 exhibited similar CD spectral profiles as those observed in Example 1 for (1, n=2+L-Ala) and (1, n=2+D-Ala), respectively.

What is claimed is:

1. A chiral nanotube, comprising a plurality of a nanotube monomers, where the nanotube monomers are arranged into a plurality of rings, each formed by hydrogen bonding between the nanotube monomers; where the rings are stacked, thereby forming a tube; and where at least a portion of the nanotube monomers include a covalently linked synthetic receptor.

2. The nanotube of claim 1, wherein each nanotube monomer is a compound having a formula selected from the group consisting of: 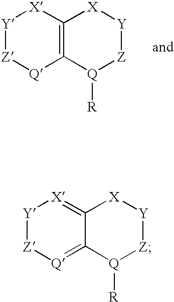
wherein:
X, X′, Y, and Y′ are each independently selected from the group consisting of hydrogen bond donors and hydrogen bond acceptors;
Z is an hydrogen bond donor, an hydrogen bond acceptor, or Z represents a single or a double bond connecting Y and Q;
Z′ is an hydrogen bond donor, an hydrogen bond acceptor, or Z′ represents a single or a double bond connecting Y′ and Q′;
Q and Q′ are each independently selected from the group consisting of —N—, —NH—, ═N—, —CH—, —CH₂—, and ═CH—; and
R is a synthetic receptor, or a derivative thereof;
providing that when X is an hydrogen bond donor, X′ is an hydrogen bond acceptor; when X is an hydrogen bond acceptor, X′ is an hydrogen bond donor; when Y is an hydrogen bond donor, Y′ is an hydrogen bond acceptor; when Y is an hydrogen bond acceptor, Y′ is an hydrogen bond donor; when Z is an hydrogen bond donor, Z′ is an hydrogen bond acceptor; and when Z is an hydrogen bond acceptor, Z′ is an hydrogen bond donor.

3. The nanotube of claim 2, wherein the hydrogen bond donor is a divalent radical having a formula selected from the group consisting of: 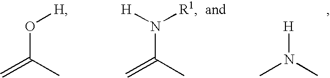
where R¹ is hydrogen or alkyl; and
the hydrogen bond acceptor is a divalent radical having a formula selected from the group consisting of:

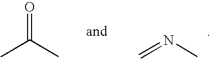

4. The nanotube of claim 1, wherein the synthetic receptor is a radical having the formula —(CH₂)ₙ—R′, where n is an integer selected from the group consisting of 2, 3, 4, and 5; and R′ is selected from the group consisting of crown ethers, cryptands, cyclodextrins, amino acids, peptides, diamines, triamines, and derivatives thereof.

5. The nanotube of claim 4, wherein R′ is selected from the group consisting of aminobenzo-18-crown-6, lysine, and 1,5-diaminopentane.

6. The nanotube of claim 1, wherein each nanotube monomer is a compound having the formula: 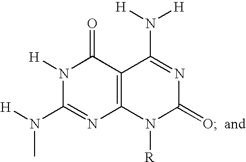
R is a synthetic receptor, or a derivative thereof.

7. A chiral nanotube comprising:
a plurality of nanotube monomers, each having a synthetic receptor; and
a plurality of chiral promoters, where the chiral promoters are bound to the synthetic receptors.

8. The nanotube of claim 7, wherein the plurality of chiral promoters is selected from the group consisting of amines and amino acids.

9. The nanotube of claim 7, wherein the plurality of chiral promoters is selected from the group consisting of amines and amino acids having a primary amine functionality.

10. The nanotube of claim 7, wherein the plurality of chiral promoters are alpha amino acids, where the alpha amino acids are substituted with alkyl, optionally-substituted aryl, optionally-substituted arylalkyl, thioalkyl, alkylthioalkyl, hydroxyalkyl, alkoxyalkyl, aminoalkyl, alkylaminoalkyl, dialkylaminoalkyl, or a group —(CH₂)ₘ—R″, where m is an integer selected from the group consisting of 1, 2, 3, 4, and 5; R″ is —CO₂R², —CONR³R⁴, or —NR⁵C(NR⁶)NR³R⁴, and R², R³, R⁴, R⁵, and R⁶ are each independently selected from the group consisting of hydrogen, alkyl, and optionally-substituted arylalkyl.

11. The nanotube of claim 7, wherein the plurality of chiral promoters is selected from the group consisting of alanine, leucine, 2-butyl-2-aminoethanoic acid, phenylalanine, 2-(naphth-2-ylmethyl)-2-aminoethanoic acid, methionine, serine, glutamic acid, and glutamine.

12. The nanotube of claim 7, wherein the plurality of chiral promoters is selected from the group consisting of alanine, leucine, phenylalanine, 2-(naphth-2ylmethyl)-2-aminoethanoic acid, and methionine.

13. The nanotube of claim 7, wherein the plurality of nanotube monomers is selected from the group of compounds consisting: 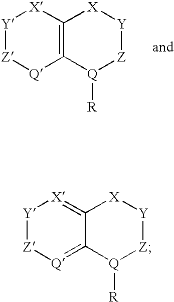
wherein:
X, X′, Y, and Y′ are each independently selected from the group consisting of hydrogen bond donors and hydrogen bond acceptors;
Z is an hydrogen bond donor, an hydrogen bond acceptor, or Z represents a single or a double bond connecting Y and Q;
Z′ is an hydrogen bond donor, an hydrogen bond acceptor, or Z′ represents a single or a double bond connecting Y′ and Q′;
Q and Q′ are each independently selected from the group consisting of —N—, —NH—, ═N—, —CH—, —CH₂—, and ═CH—; and
R is a synthetic receptor, or a derivative thereof;
providing that when X is an hydrogen bond donor, X′ is an hydrogen bond acceptor; when X is an hydrogen bond acceptor, X′ is an hydrogen bond donor; when Y is an hydrogen bond donor, Y′ is an hydrogen bond acceptor; when Y is an hydrogen bond acceptor, Y′ is an hydrogen bond donor; when Z is an hydrogen bond donor, Z′ is an hydrogen bond acceptor; and when Z is an hydrogen bond acceptor, Z′ is an hydrogen bond donor.

14. The nanotube of claim 13, wherein the hydrogen bond donor is a divalent radical having a formula selected from the group consisting of: 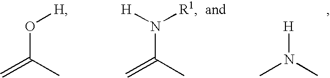
where R′ is hydrogen or alkyl; and
the hydrogen bond acceptor is a divalent radical having a formula selected from the group consisting of:

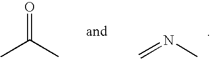

15. The nanotube of claim 13, wherein R is a synthetic receptor having the formula —(CH₂)ₙ—R′, where n is an integer selected from the group consisting of 2, 3, 4, and 5, and R′ is selected from the group consisting of crown ethers, cryptands, cyclodextrins, amino acids, peptides, diamines, triamines, and derivatives thereof.

16. The nanotube of claim 15, wherein R′ is selected from the group consisting of aminobenzo- 18-crown-6, lysine, and 1,5-diaminopentane.

17. The nanotube of claim 7, wherein each nanotube monomer is a compound having the formula: 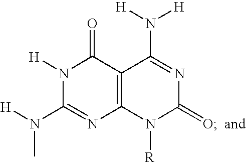
R is the synthetic receptor or a derivative thereof.

18. A process for forming an optically active solution of chiral nanotubes comprising, adding a promoter to a solution of nanotubes where the nanotubes are assembled from nanotube monomers at least a portion of which include a synthetic receptor, in an amount effective to cause an enantiomeric excess of one chiral nanotube.

19. The process of claim 18, wherein the adding step includes adding a solution of the promoter to the solution of nanotubes, where the solution of the promoter is optically active and the solution of nanotubes is a racemic mixture of chiral nanotubes.

20. The process of claim 18, wherein the adding step includes adding the promoter to the solution of nanotubes, where the solution of nanotubes includes a first chiral nanotube and a second chiral nanotube, where the first and second chiral nanotubes have opposite chirality, and the promoter is added in an amount effective to convert at least a portion of the chirality of the first chiral nanotube into the chirality of the second chiral nanotube.

21. A process for forming a dilute solution of chiral nanotubes comprising, adding a promoter to a solution of nanotube monomers in an amount effective to cause the formation of the chiral nanotubes.

22. The process of claim 21, wherein the adding step includes adding a solution of the promoter, where the solution of the promoter is optically active.

23. The process of claim 21, wherein the adding step includes adding an homochiral promoter.

24. A process for stabilizing a solution of nanotubes to dilution comprising:
(a) adding a promoter to a solution of nanotubes in an amount effective to prevent the disassembly of the nanotubes during dilution over a pre-determined range of concentration; and
(b) diluting the solution of nanotubes over the pre-determined range of concentration.

25. The process of claim 24, wherein the adding step includes adding an homochiral promoter to the solution of nanotubes in an amount effective to preferentially prevent the disassembly of the one nanotube enantiomer relative to the other nanotube enantiomer during dilution, thereby stabilizing an optically active dilute solution of chiral nanotubes during dilution.

26. A compound having the formula: 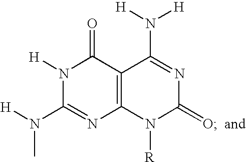
wherein:
R is a radical having the formula —(CH₂)ₙ—R′, where n is an integer selected from the group consisting of 2, 3, 4, and 5; and R′ is selected from the group consisting of crown ethers, cryptands, cyclodextrins, peptides, diamines, triamines, and derivatives thereof.

27. The compound of claim 26, wherein R is a radical having the formula —(CH₂)ₙ—R′, where n is an integer selected from the group consisting of 2, 3, 4, and 5; and R′ is selected from the group consisting of crown ethers, and derivatives thereof.
